(19) United States
(12) Patent Application Publication
(10) Pub. No.: US 2007/0208411 A1
(43) Pub. Date: Sep. 6, 2007

(54) Bifurcated stent with surface area gradient

(75) Inventors: Michael P. Meyer, Richfield, MN (US); Thomas E. Broome, Shakopee, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 11/368,965

(22) Filed: Mar. 6, 2006

(51) Int. Cl. A61F 2/06

(52) U.S. Cl. 623/1.15

(57) ABSTRACT

A stent may comprise a plurality of interconnected strut members defining a plurality of serpentine bands and a plurality of connector struts. Adjacent serpentine bands may be connected by at least one connector strut. The stent may further comprise an outwardly deployable side branch structure. The total amount of surface area of structural strut members in a given unit area on the surface of the stent is higher in areas near the side branch structure than in areas spaced away from the side branch structure.

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

There remains a need for stents designed to be deployed at a bifurcation that provide increased amounts of support to both the primary and side branch vessels.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In at least one embodiment, the invention is directed to a stent comprising a plurality of interconnected strut members that define a plurality of cells. A portion of the interconnected strut members comprise a side branch structure defining a side branch cell that is shaped differently than other cells of the stent. The stent comprises a first end portion, a middle portion and a second end portion. The side branch structure is located in the middle portion of the stent. The interconnected strut members further define a plurality of serpentine bands and a plurality of connector struts. Adjacent serpentine bands are connected by at least one connector strut. A first unit area located in the middle portion of the stent includes at least two interconnected strut members. A second unit area located outside of the middle portion of the stent has a size and shape similar to the first unit area. The first unit area includes more metal than the second unit area. In some embodiments, a total outer surface area of the interconnected strut members in the first unit area is greater than a total outer surface area of the interconnected strut members in the second unit area.

In at least one other embodiment, the invention is directed to a stent comprising a plurality of interconnected strut members that define a plurality of cells. A portion of the interconnected strut members comprise a side branch structure defining a side branch cell that is shaped differently than other cells of the stent. The stent comprises a first end portion, a middle portion and a second end portion. The side branch structure is located in the middle portion of the stent. The interconnected strut members further define a plurality of serpentine bands and a plurality of connector struts. Adjacent serpentine bands are connected by at least one connector strut. A first serpentine band located in the middle portion of the stent is connected to a second serpentine band by a first connector column comprising a plurality of connector struts. A third serpentine band located outside the middle portion of the stent is connected to a fourth serpentine band by a second connector column comprising at least one connector strut. The first connector column has more connector struts than the second connector column.

In at least one other embodiment, the invention is directed to a stent comprising a plurality of interconnected strut members that define a plurality of cells. A portion of the interconnected strut members comprise a side branch structure defining a side branch cell that is shaped differently than other cells of the stent. The stent comprises a first end portion, a middle portion and a second end portion. The side branch structure is located in the middle portion of the stent. The interconnected strut members further define a plurality of serpentine bands and a plurality of connector struts. Adjacent serpentine bands are connected by at least one connector strut. A first serpentine band located in the middle portion of the stent comprises a first strut having a first width. A second serpentine band located outside of the middle portion of the sent comprises a second strut having a second width, the first width being greater than the second width.

In at least one other embodiment, the invention is directed to a stent comprising a plurality of interconnected strut members that define a plurality of cells. A portion of the interconnected strut members comprise a side branch structure defining a side branch cell that is shaped differently than other cells of the stent. The stent comprises a first end portion, a middle portion and a second end portion. The side branch structure is located in the middle portion of the stent. The interconnected strut members further define a plurality of serpentine bands and a plurality of connector struts. Adjacent serpentine bands are connected by at least one connector strut. The stent may further comprise a first serpentine band and a second serpentine band. Adjacent distal valleys of the first serpentine band are separated by a first distance. Adjacent distal valleys of the second serpentine band are separated by a second distance that is greater than the first distance.

In at least one other embodiment, the invention is directed to a stent comprising a plurality of interconnected strut members that define a plurality of cells. A portion of the interconnected strut members comprise a side branch structure defining a side branch cell that is shaped differently than other cells of the stent. The stent comprises a first end portion, a middle portion and a second end portion. The side branch structure is located in the middle portion of the stent. The interconnected strut members further define a plurality of serpentine bands and a plurality of connector struts. Adjacent serpentine bands are connected by at least one connector strut. The stent may further comprise a first serpentine band and a second serpentine band. The first serpentine band is located in the middle portion of the stent and comprises a first component length. The second serpentine band is located distal to the first serpentine band. The second serpentine band comprises a second component length that is greater than the first component length.

In at least one other embodiment, the invention is directed to a stent comprising a plurality of interconnected strut members that define a plurality of cells. A portion of the interconnected strut members comprise a side branch structure defining a side branch cell that is shaped differently than other cells of the stent. The stent comprises a first end portion, a middle portion and a second end portion. The side branch structure is located in the middle portion of the stent. The interconnected strut members further define a plurality of serpentine bands and a plurality of connector struts. Adjacent serpentine bands are connected by at least one connector strut. The stent may further comprise a first serpentine band and a second serpentine band. The first serpentine band is located in the middle portion of the stent and comprises a first component length. The second serpentine band is located distal to the first serpentine band. The second serpentine band comprises a second component length that is greater than the first component length. The second serpentine band further includes fewer distal valleys than the first serpentine band.

In some other embodiments, the invention is directed to methods of making a stent having a strut pattern according to the embodiments described herein.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described a embodiments of the invention.

### BRIEF DESCRIPTION OF THE SEVERAL VIEWS OF THE DRAWING(S)

A detailed description of the invention is hereafter described with specific reference being made to the drawings.

FIG. 1 shows an embodiment of a pattern for a stent.

FIG. 2 shows another embodiment of a pattern for a stent.

FIG. 3 shows another embodiment of a pattern for a stent.

FIG. 4 shows another embodiment of a pattern for a stent.

FIG. 5 shows another embodiment of a pattern for a stent.

FIG. 6 shows another embodiment of a pattern for a stent.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

In some embodiments, the invention is directed to stents having a side branch structure, wherein the total amount of surface area of structural struts in a given unit area on the surface of the stent is higher in areas near the side branch structure than in areas spaced away from the side branch structure. These designs may allow for increased drug delivery, vessel support, scaffolding and radial strength to portions of a bifurcated vessel that are typically affected with disease. The increased concentration of structural struts around the side branch structure also provides a better support anchor for the main branch structure of the stent, helping to prevent the main branch structure from being pushed into the side branch vessel as the stent is deployed. The increased concentration of structural struts around the side branch structure also provides a better transition between the relatively flexible main branch structure and the generally stiffer side branch structure.

In some embodiments, the invention is directed to stent designs that comprise side branch structure, which may include a plurality of outwardly deployable side branch petals. The stents may further comprise a plurality of structural struts, and openings of the stent located between the struts may comprise cells. Anywhere along the stent, a unit area of space may be defined on the surface of the stent. The unit area may be oriented over struts and cells. Thus, a portion of the unit area may comprise strut surface area, and a portion of the unit area may comprise cell surface area.

A plurality of unit areas may be defined on a stent, wherein each unit area comprises the same size and shape. The strut surface area in unit areas located near the side branch structure may be higher than the strut surface area in unit areas spaced apart from the side branch structure. In some embodiments, the strut surface area per unit area may decrease as the unit area is moved from the side branch structure outwardly along the length of the stent toward either end of the stent.

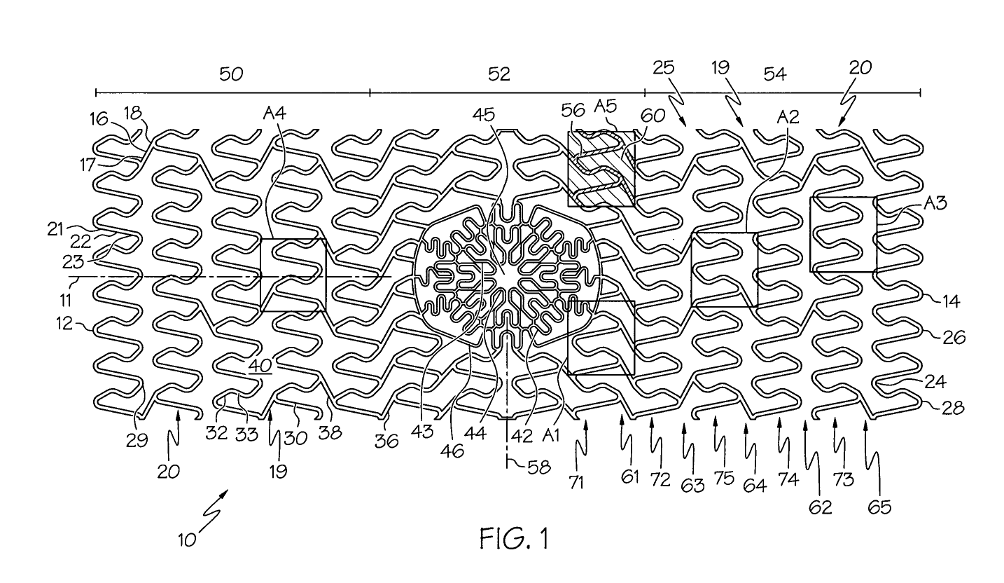

FIGS. 1-6 show various embodiments of flat patterns for a stent 10. Each stent 10 pattern may have a proximal end 12 and a distal end 14, and may comprise a plurality of serpentine bands 20. Each serpentine band 20 may comprise a plurality of struts 22, each strut 22 having a first or proximal end 21 and a second or distal end 23. Circumferentially adjacent struts 22 within a serpentine band 20 may be connected by turns 28. Turns 28 located on a proximal side of a serpentine band 20 may comprise proximal peaks 24, and turns 28 located on a distal side of a serpentine band 20 may comprise distal valleys 26.

The struts 22 of a serpentine band 20 may comprise straight struts 30 and/or bent struts 32. A straight or linear strut 30 may be substantially straight along its length. A bent strut 32 may include curvature along its length. In some embodiments, a bent strut 32 may comprise an s-shape and may include an inflection point 33 wherein the curvature changes orientation.

In some embodiments, a serpentine band 20 may comprise alternating straight struts 30 and bent or s-shaped struts 32. Each straight strut 30 may be oriented between two bent struts 32. Thus, a first end 21 of a straight strut 30 may be connected to a proximal peak 24 which may connect to a bent strut 32 located on one side of the straight strut 30. A second end 23 of the straight strut 30 may be connected to a distal valley 26 which may connect to another bent strut 32 located on the other side of the straight strut 30. Similarly, each bent strut 32 may be oriented between two straight struts 30. Thus, a first end 21 of a bent strut 32 may be connected to a proximal peak 24 which may connect to a straight strut 30 located on one side of the bent strut 32. A second end 23 of the bent strut 32 may be connected to a distal valley 26 which may connect to another straight strut 30 located on the other side of the bent strut 32.

Each strut 22 may have a width. In some embodiments, all struts 22 may have the same width. In some embodiments, bent struts 32 may have a different width than straight struts 30. In various other embodiments, individual struts 22 may each have any suitable width dimension.

Serpentine bands 20 which are adjacent to one another along the length of the stent 10 may be connected by at least one connector strut 16. Connector columns 19 may be oriented between adjacent serpentine bands 20. Each connector column 19 may comprise at least one and in some embodiments a plurality of connector struts 16. Connector struts 16 may connect to any portion of a serpentine band 20, such as a turn 28, or in some embodiments, a strut 22. In some embodiments, a connector strut 16 may span between turns 28 of adjacent serpentine bands 20. For example, a first end 17 of a connector strut 16 may connect to a distal valley 26 of one serpentine band 20, and a second end 18 of the connector strut 16 may connect to a proximal peak 24 of an adjacent serpentine band 20.

In some embodiments, a connector strut 16 may be linear or straight along its length. In some embodiments, a connector strut 16 may have curvature along its length.

In some embodiments, a stent 10 may comprise a first type of connector strut 36 and a second type of connector strut 38. A first connector strut 36 may extend in a first direction. The first connector strut 36 may be oriented at a first angle to a stent lengthwise axis 11. A second connector strut 38 may extend in a second direction that is different than or non-parallel to the first direction. Therefore, a second connector strut 38 may be oriented at a second angle to a stent lengthwise axis 11, the second angle being different than the first angle. In some embodiments, the first angle and the second angle may have the same magnitude but different orientations. For example, a first connector strut 36 may form a 70° angle with a stent lengthwise axis 11, while a second connector strut 38 may form a negative 70° angle with the stent lengthwise axis 11. In some embodiments, a first angle may comprise a mirror image of a second angle across a stent lengthwise axis 11.

In some embodiments, all of the first connector struts 36 of the stent 10 may be parallel to one another. In some embodiments, a first connector strut 36 may extend between turns 28 which connect a straight strut 30 to a bent strut 32. In some embodiments, each side of the first connector strut 36 may extend from a turn 28 in the direction of the side of the turn 28 which connects to a bent strut 32.

In some embodiments, all of the second connector struts 38 of the stent 10 may be parallel to one another. In some embodiments, a second connector strut 38 may extend between turns 28 which connect a straight strut 30 to a bent strut 32. In some embodiments, each side of the second connector strut 38 may extend from a turn 28 in the direction of the side of the turn 28 which connects to a straight strut 30.

Each serpentine band 20 may include unconnected turns 29 from which no connector strut 16 extends.

A stent 10 may have any suitable number of serpentine bands 20. A stent 10 may have any suitable number of struts 22 per serpentine band 20. A stent 10 may further have any suitable number of connector struts 16 extending between adjacent serpentine bands 20.

A stent 10 may comprise a plurality of cells 40. A cell 40 may comprise an opening in the stent 10 wall portion between serpentine bands 20 and connector struts 16. In some embodiments, a cell 40 may be bounded by a serpentine band 20, a connector strut 16, another serpentine band 20 and another connector strut 16.

A stent 10 may further comprise a side branch structure 42 having a plurality of outwardly deployable petals 44. The side branch structure 42 may comprise a plurality of side branch struts 43, and in some embodiments may include a continuous peripheral side branch strut 46 that extends about other elements of the side branch structure 42. The side branch structure 42 may define at least one side branch cell 45, which may be different in size and/or shape from cells 40 of the stent 10 that are located outside of the side branch structure 42.

In some embodiments, it is desirable for areas surrounding the side branch structure 42 to have a greater density of structural strut members 22 than are provided in locations that are spaced from the side branch structure 42, such as the ends 12, 14 of the stent 10.

In some embodiments, a stent 10 may comprise a first end portion 50, a middle portion 52 and a second end portion 54. Each portion 50, 52, 54 may have the same length as measured along a stent lengthwise axis 11. In some embodiments, a portion of the side branch structure 42 or all of the side branch structure 42 may be located in the middle portion 52.

A unit area, for example unit area A1, may be defined anywhere on the stent 10. A unit area may have any suitable size and shape, and may include any number of structural elements, such as struts 22 and connector struts 16. A unit area may further include void space or cells 40, 45. A comparison or ratio of structural element area to cell area may be defined for any unit area. A comparison or ratio of structural element area to total area may be defined for any unit area. Multiple unit areas that are similar in size and shape may be compared, and may have varying amounts of structural element area.

A first unit area A1 may be defined near the side branch structure 42. At least a portion of the first unit area A1 may be located in the middle portion 52 of the stent 10. In some embodiments, the entire first unit area A1 may be located in the middle portion 52 of the stent 10. In some embodiments, a first unit area A1 may include at least one element of the side branch structure 42. In some embodiments, a first unit area does not include any of the side branch structure 42, for example as shown by unit area A5.

A second unit area A2 may be defined on the stent 10. The second unit area A2 may have the same size and shape as the first unit area A1. At least a portion of the second unit area A2 may be oriented outside of the middle portion 52 of the stent 10, such as in the first end portion 50 or in the second end portion 54, for example as shown in FIG. 1. The second unit area A2 may be located farther away from the side branch structure 42 than the first unit area A1.

Each unit area may have a total structural element area 56 and a total cell area 60. The total structural element area 56 may comprise the total surface area of all structural elements of the stent 10 located in the unit area as measured on the outer surface of the stent 10. The total cell area 60 may comprise the total area of voids or cells 40, 45 located in the unit area as measured on the outer surface of the stent 10. The total structural element area 56 combined with the total cell area 60 may equal the total area of the unit area.

The first unit area A1 may have a greater amount of structural element area 56 than the second unit area A2. The first unit area A1 may have a lesser amount of cell area 60 than the second unit area A2. The first unit area A1 may have more metal than the second unit area A2.

In some embodiments, unit areas that are being compared may be placed over portions of the stent 10 having similar characteristics. For example, referring to FIG. 1, in some embodiments, a proximal lower corner of a first unit area A1 may be oriented over a proximal peak 24 that connects to a straight strut 30 that extends into the first unit area A1. Similarly, for a second unit area A2, and a third unit area A3, the corresponding proximal lower corner of the unit area A2, A3 may be oriented over a proximal peak 24 that connects to a straight strut 30 that extends into the unit area A2, A3.

A third unit area A3 may be defined on the stent 10. The third unit area A3 may have the same size and shape as the first unit area A1. The third unit area A3 may be oriented outside of the middle portion 52 of the stent 10, such as in the first end portion 50 or in the second end portion 54 as shown in FIG. 1. The third unit area A3 may be located farther away from the side branch structure 42 than the second unit area A2.

The second unit area A2 may have a greater amount of structural element area 56 than the third unit area A3. The second unit area A2 may have a lesser amount of cell area 60 than the third unit area A3. The second unit area A2 may have more metal than the third unit area A3. Therefore, the first unit area A1 may have a greater amount of structural element area 56 than the third unit area A3. The first unit area A1 may have a lesser amount of cell area 60 than the third unit area A3.

A fourth unit area A4 may be defined on the stent 10. The fourth unit area A4 may have the same size and shape as the first unit area A1. The fourth unit area A4 may be located farther away from the side branch structure 42 than the first unit area A1. The first unit area A1 may have a greater amount of structural element area 56 than the fourth unit area A4. The first unit area A1 may have a lesser amount of cell area 60 than the fourth unit area A4.

In some embodiments, the fourth unit area A4 may be located across the side branch structure 42 from the second unit area A2. In some embodiments, the structural elements of the stent 10 located in the fourth unit area A4 may comprise a mirror image of the structural elements of the stent 10 located in the second unit area A2. The mirror image may be taken across a mirror image line 58 that passes through the center or centroid of the side branch structure 42. In some embodiments, a mirror image line 58 may comprise a circumference of the stent 10. In some embodiments, a mirror image line 58 may bisect the stent 10 along its length.

Various other unit areas may be defined anywhere on the stent 10. In general, when comparing two unit areas of similar size and shape, the unit area that is located closer to the side branch structure 42 may have more structural element area 56 and less cell area 60 than the unit area that is located farther away from the side branch structure 42. The unit area that is located closer to the side branch structure 42 may have more metal than the unit area that is located farther away from the side branch structure 42. Distance from the side branch structure 42 may be measured from the center of the side branch structure 42 to the center of the respective unit area.

In some embodiments, the invention is directed to a stent 10 wherein the number of connectors 16 per connector column 19 may decrease as the distance from the side branch structure 42 increases, for example as shown in FIG. 1.

A full connector column 25 may comprise a connector column 19 that extends fully about a circumference of the stent 10. A full connector column 25 is not interrupted by side branch structure 42.

Referring to FIG. 1, a first connector column 61 may be located between a first serpentine band 71 and a second serpentine band 72. The first serpentine band 71 may be located in the middle portion 52 of the stent 10. The first connector column 61 may be located in the middle portion 52 of the stent 10. A second connector column 62 may be located between a third serpentine band 73 and a fourth serpentine band 74. The third serpentine band 73 may be located outside of the middle portion 52, for example being located in the second end portion 54. The first connector column 61 may have a greater number of connector struts 16 than the second connector column 62.

In some embodiments, the first connector column 61 may comprise a full connector column 25. The first connector column 61 may be adjacent to the side branch structure 42 along the length of the stent 10. The first connector column 61 may be located closer to the side branch structure 42 than the second connector column 62 along the length of the stent 10.

The stent 10 may further comprise a fifth serpentine band 75. The fifth serpentine band 75 may be adjacent to the second serpentine band 72 along the length of the stent and may be connected to the second serpentine band 72 by a third connector column 63. The first connector column 61 may have a greater number of connector struts 16 than the third connector column 63. The third connector column 63 may have more connector struts 16 than the second connector column 62.

The fifth serpentine band 75 may further be connected to the fourth serpentine band 74 by a fourth connector column 64. The fourth connector column 64 may be located outside of the middle portion 52. The third connector column 63 may have more connector struts 16 than the fourth connector column 64. The fourth connector column 64 may have more connector struts 16 than the second connector column 62.

The stent 10 may further comprise a fifth connector column 65, which may comprise at least one connector strut 16 that is connected to the third serpentine band 73. In some embodiments, the fifth connector column 65 may have the same number of connector struts 16 as the second connector column 62. In some embodiments, the fifth connector column 65 may be the closest connector column 19 to the end of the stent 14.

In some embodiments, the first connector column 61 may be located distal to the side branch structure 42 along the length of the stent 10, and each connector column 19 located distal to the first connector column 61 includes less connector struts 16 than the first connector column 61. In some embodiments, each connector column 19 located distal to the first connector column 61 may include less connector struts 16 than the previous connector column 19 as the stent 10 is traversed toward the distal end 14. For example, for any selected connector column 19 located distal to the side branch structure 42 along the length of the stent 10, another connector column 19 located distal to the selected connector column 19 may have less connector struts 16 than the selected connector column 19, and another connector column 19 located proximal to the selected connector column 19 may have more connector struts 16 than the selected connector column 19. In some embodiments, the decrease in the number of connector struts 16 per connector column 19 may be uniform as the stent 10 is traversed from a connector column 19, toward an end 12, 14 of the stent 10 in a direction away from the side branch structure 42.

In some embodiments, the connector columns 61-65 and serpentine bands 71-75 as described with respect to FIG. 1 may all be located distal to the side branch structure 42 along the length of the stent 10. As shown in FIG. 1, the stent structure on the proximal side of the side branch structure 42 may comprise a substantial mirror image of the stent structure on the distal side of the side branch structure 42. The mirror image may be taken across a mirror image line 58 that passes through the center of the side branch structure 42. Therefore, the number of connector struts 16 per connector column 19 may decrease on either side of the side branch structure 42 from a connector column 19 adjacent to the side branch structure 42 towards the respective end 12, 14 of the stent 10.

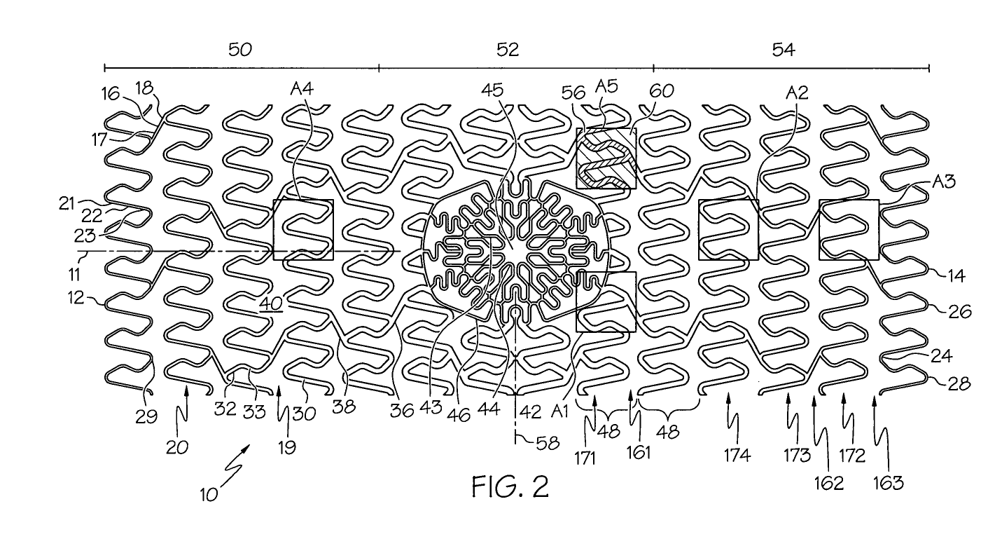

In some embodiments, the invention is directed to a stent 10 wherein the width of serpentine bands 20 and/or connector struts 16 may decrease as the distance away from the center of the side branch structure 42 along the length of the stent 10 increases, for example as shown in FIG. 2.

Referring to FIG. 2, a first serpentine band 171 may be located in the middle portion 52 of the stent 10 and may comprise a strut 22 having a first width. A second serpentine band 172 may be located outside of the middle portion 52, for example being located in the second end portion 54, and may comprise a strut 22 having a second width. The first width may be greater than the second width. The first serpentine band 171 may be located closer to the side branch structure 42 than the second serpentine band 172.

A third serpentine band 173 may be located between the first serpentine band 171 and the second serpentine band 172. The third serpentine band 173 may comprise a strut 22 having a third width. The third width may be less than the first width. The third width may be greater than the second width.

A fourth serpentine band 174 may be located between the first serpentine band 171 and the third serpentine band 173. The fourth serpentine band 174 may comprise a strut 22 having a fourth width. The fourth width may be less than the first width. The fourth width may be greater than the third width.

In some embodiments, a first connector column 161 may comprise at least one connector strut 16 having a first connector strut width, the connector strut 16 being connected to the first serpentine band 171. A second connector column 162 may comprise at least one connector strut 16 having a second connector strut width, the connector strut 16 being connected to the second serpentine band 172. The first connector strut width may be greater than the second connector strut width.

The stent 10 may further comprise a third connector column 163 including a connector strut 16 having a third connector strut width, the connector strut 16 being connected to the second serpentine band 172. The third connector strut width may be less than the second connector strut width.

In some embodiments, the first serpentine band 171 may be located distal to the side branch structure 42, and each serpentine band 20 that is located distal to the first serpentine band 171 may comprise a strut having a width that is less than the first width.

In some embodiments, the width of struts 22 of serpentine bands 20 may decrease as the stent 10 is traversed from the center of the side branch structure 42 toward either end 12, 14 of the stent 10. In some embodiments, the width of struts 22 of serpentine bands 20 may continuously or uniformly decrease as the stent 10 is traversed from the center of the side branch structure 42 toward either end 12, 14 of the stent 10.

In some embodiments, the width of connector struts 16 of connector columns 19 may decrease as the stent 10 is traversed from the center of the side branch structure 42 toward either end 12, 14 of the stent 10. In some embodiments, the width of connector struts 16 of connector columns 19 may decrease continuously or uniformly as the stent 10 is traversed from the center of the side branch structure 42 toward either end 12, 14 of the stent 10.

In some embodiments, the width of connector struts 16 of connector columns 19 and the width of struts 22 of serpentine bands 20 may decrease as the stent 10 is traversed from the center of the side branch structure 42 toward either end 12, 14 of the stent 10. In some embodiments, the decrease may be continuous or uniform.

In some embodiments, a connector column 19 may comprise connector struts 16 that have the same width as struts 22 of an adjacent serpentine band 20. When the widths are the same, the connector column 19 and serpentine band 20 may be considered a width pair 48. For example, the first connector column 161 may comprise connector struts 16 that have the same width as the struts 22 of the first serpentine band 171. The first connector column 161 and first serpentine band 171 may comprise a width pair 48. In some embodiments, the widths of adjacent width pairs 48 may decrease as the stent 10 is traversed from the center of the side branch structure 42 toward an end 12, 14 of the stent 10. In some embodiments, the decrease in the width of elements of the width pairs 48 between adjacent width pairs 48 may be continuous or uniform as the stent 10 is traversed from the center of the side branch structure 42 toward an end 12, 14 of the stent 10.

In some embodiments, the connector columns 161-163 and serpentine bands 171-174 as described with respect to FIG. 2 may all be located distal to the center of the side branch structure 42 along the length of the stent 10. In some embodiments, the stent structure on the proximal side of the center of the side branch structure 42 may include similar features. As shown in FIG. 2, the stent structure on the proximal side of the center of the side branch structure 42 may comprise a substantial mirror image of the stent structure on the distal side of the side branch structure 42. The mirror image may be taken across a mirror image line 58 that passes through the center of the side branch structure 42.

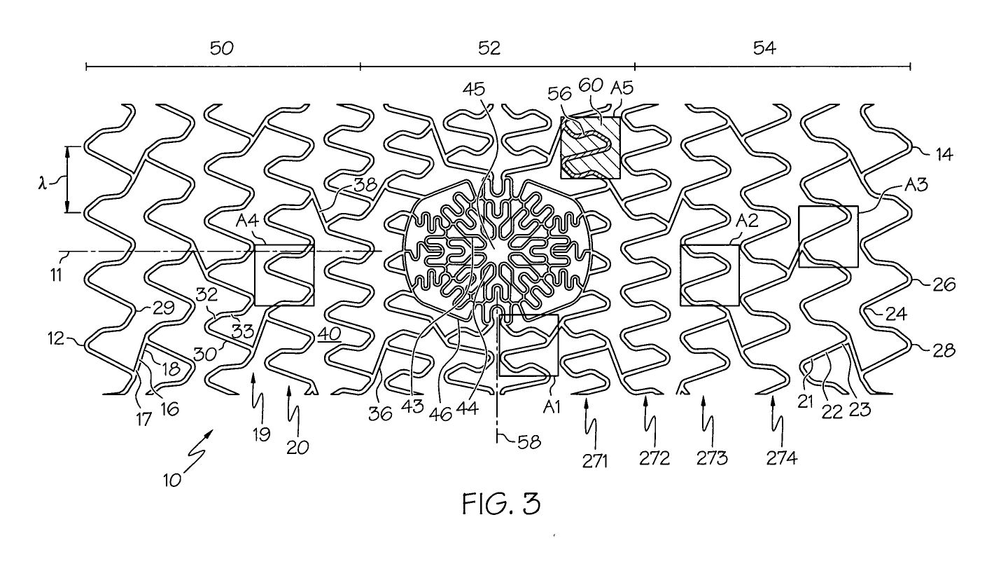

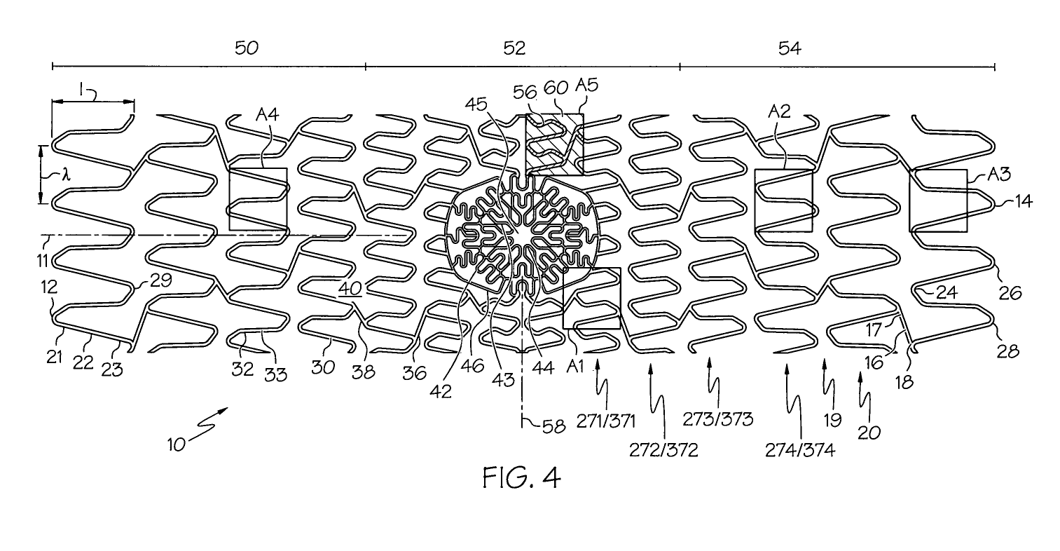

In some embodiments, the invention is directed to a stent 10 wherein the wavelength λ of serpentine bands 20 may increase as the distance away from the center of the side branch structure 42 along the length of the stent 10 increases, for example as shown in FIGS. 3 and 4.

Referring to FIGS. 3 and 4, each serpentine band 20 may comprise a substantially wave-like shape. Each serpentine band 20 may have a wavelength λ or distance between repeating elements of the serpentine band 20. For example, a wavelength λ may comprise a distance between adjacent proximal peaks 24 of a serpentine band 20, or a distance between adjacent distal valleys 26 of a serpentine band 20. In some embodiments, the wavelength λ may be measured about a circumference of the stent 10.

In some embodiments, a first serpentine band 271 may comprise a first wavelength λ, wherein adjacent distal valleys 26 of the first serpentine band 271 are separated by a first distance. The first serpentine band 271 may be located in the middle portion 52 of the stent 10, and in some embodiments may connect to at least one element of the side branch structure 42. A second serpentine band 272 may comprise a second wavelength λ, wherein adjacent distal valleys 26 of the second serpentine band 272 are separated by a second distance. The second serpentine band 272 may be located farther away from the center of the side branch structure 42 along the length of the stent 10 than the first serpentine band 271. The second wavelength λ may be greater than the first wavelength λ, and the second distance may be greater than the first distance.

In some embodiments, the first serpentine band 271 may be located distal to the center of the side branch structure 42, and the second serpentine band 272 may be located distal to the first serpentine band 271.

A third serpentine band 273 may comprise a third wavelength λ, wherein adjacent distal valleys 26 of the third serpentine band 273 are separated by a third distance. The third serpentine band 273 may be located outside of the middle portion 52 of the stent 10, for example being located in the second end portion 54. The third serpentine band 273 may be located distal to both the first serpentine band 271 and the second serpentine band 272. The third wavelength λ may be greater than both the first and second wavelengths λ, and the third distance may be greater than both the first and second distances. In some embodiments, the third serpentine band 273 may have fewer distal valleys 26 than the second serpentine band 272.

In some embodiments, a stent 10 may further comprise a fourth serpentine band having a fourth wavelength λ, wherein adjacent distal valleys 26 of the fourth serpentine band 274 are separated by a fourth distance. The fourth serpentine band 274 may be located outside of the middle portion 52 of the stent 10, for example being located in the second end portion 54. The fourth serpentine band 274 may be located distal to the third serpentine band 273. The fourth wavelength λ may be greater than the third wavelength λ, and the fourth distance may be greater than the third distance. In some embodiments, the fourth serpentine band 274 may have fewer distal valleys 26 than the third serpentine band 273.

In some embodiments, the wavelength λ of serpentine bands 20 may increase as the stent 10 is traversed from the center of the side branch structure 42 toward either end 12, 14 of the stent 10. In some embodiments, the wavelength λ of serpentine bands 20 may continuously or uniformly increase between adjacent serpentine bands 20 as the stent 10 is traversed from the center of the side branch structure 42 toward either end 12, 14 of the stent 10.

In some embodiments, the serpentine bands 271-274 as described with respect to FIGS. 3 and 4 may all be located distal to the center of the side branch structure 42 along the length of the stent 10. In some embodiments, the stent structure on the proximal side of the center of the side branch structure 42 may include similar features. As shown in FIGS. 3 and 4, the stent structure on the proximal side of the center of the side branch structure 42 may comprise a substantial mirror image of the stent structure on the distal side of the side branch structure 42. The mirror image may be taken across a mirror image line 58 that passes through the center of the side branch structure 42.

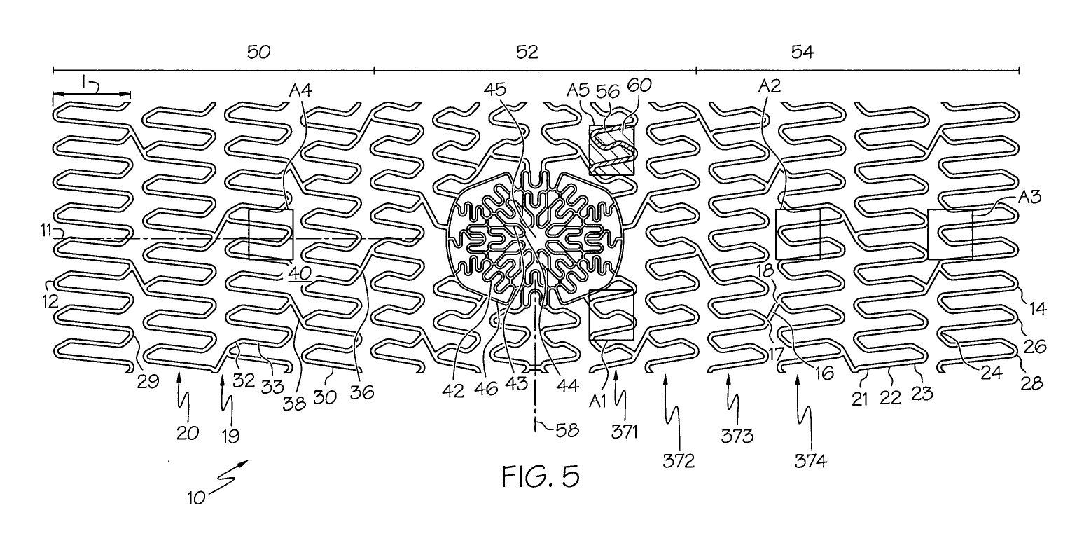

In some embodiments, the invention is directed to a stent 10 wherein the length of struts 22 of a serpentine band 20 and/or the stent lengthwise distance spanned by a serpentine band 20 increases as the distance away from the center of the side branch structure 42 along the length of the stent 10 increases, for example as shown in FIGS. 4 and 5.

Referring to FIGS. 4 and 5, each serpentine band 20 may have a component length l comprising a distance spanned by the serpentine band 20 as measured in a stent lengthwise direction, which may be parallel to the stent lengthwise axis 11.

A first serpentine band 371 may have a first component length as measured in a stent lengthwise direction. The first serpentine band 371 may be located in the middle portion 52 of the stent 10, and in some embodiments may connect to at least one element of the side branch structure 42. A second serpentine band 372 may have a second component length as measured in a stent lengthwise direction. The second serpentine band 372 may be located farther away from the center of the side branch structure 42 along the length of the stent 10 than the first serpentine band 371. The second component length may be greater than the first component length. Struts 22 of the second serpentine band 372 may be longer than struts 22 of the first serpentine band 371.

In some embodiments, a stent 10 may further comprise a third serpentine band 373 having a third component length as measured in a stent lengthwise direction. The third serpentine band 373 may be located outside of the middle portion 52 of the stent 10, for example being located in the second end portion 54. The third serpentine band 373 may be located farther away from the center of the side branch structure 42 along the length of the stent 10 than the second serpentine band 372. The third component length may be greater than the second component length. Struts 22 of the third serpentine band 373 may be longer than struts 22 of the second serpentine band 372.

In some embodiments, a stent 10 may further comprise a fourth serpentine band 374 having a fourth component length as measured in a stent lengthwise direction. The fourth serpentine band 374 may be located outside of the middle portion 52 of the stent 10, for example being located in the second end portion 54. The fourth serpentine band 374 may be located farther away from the center of the side branch structure 42 along the length of the stent 10 than the third serpentine band 373. The fourth component length may be greater than the third component length. Struts 22 of the fourth serpentine band 374 may be longer than struts 22 of the third serpentine band 373.

In some embodiments, the component length l of various serpentine bands 20 as measured in a stent lengthwise direction may increase as the stent 10 is traversed from the center of the side branch structure 42 toward either end 12, 14 of the stent 10. In some embodiments, the component length l of various serpentine bands 20 may continuously or uniformly increase between adjacent serpentine bands 20 as the stent 10 is traversed from the center of the side branch structure 42 toward either end 12, 14 of the stent 10.

In some embodiments, the length of struts 22 of various serpentine bands 20 may increase as the stent 10 is traversed from the center of the side branch structure 42 toward either end 12, 14 of the stent 10. In some embodiments, the length of struts 22 of various serpentine bands 20 may continuously or uniformly increase between adjacent serpentine bands 20 as the stent 10 is traversed from the center of the side branch structure 42 toward either end 12, 14 of the stent 10.

In some embodiments, the serpentine bands 371-374 as described with respect to FIGS. 4 and 5 may all be located distal to the center of the side branch structure 42 along the length of the stent 10. In some embodiments, the stent structure on the proximal side of the center of the side branch structure 42 may include similar features. As shown in FIGS. 4 and 5, the stent structure on the proximal side of the center of the side branch structure 42 may comprise a substantial mirror image of the stent structure on the distal side of the side branch structure 42. The mirror image may be taken across a mirror image line 58 that passes through the center of the side branch structure 42.

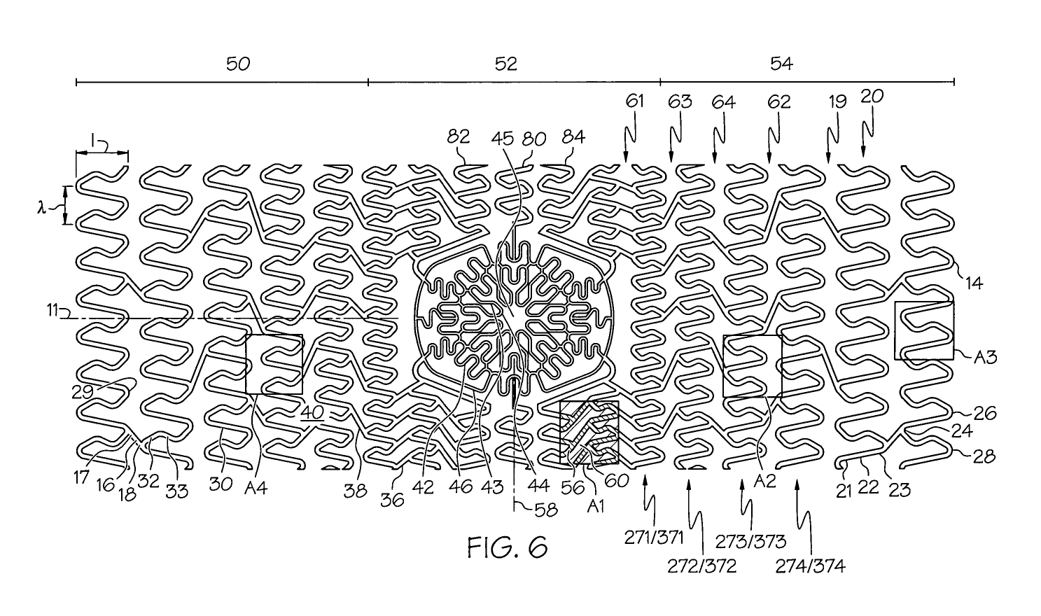

FIG. 6 shows another embodiment of a stent 10 wherein the concentration of structural elements may be greater in areas near the side branch structure 42. The stent 10 of FIG. 6 includes various features of other embodiments described herein with respect to FIGS. 1-5, as indicated by the use of like reference numerals.

The number of connectors 16 per connector column 19 may decrease as the distance from the side branch structure 42 increases, for example as described herein with respect to FIG. 1. Reference numerals 61-64 indicate connector columns 19 wherein the description of the embodiment of FIG. 1 may be applied to the embodiment of FIG. 6.

The wavelength λ of serpentine bands 20 may increase as the distance away from the center of the side branch structure 42 along the length of the stent 10 increases, for example as described herein with respect to FIGS. 3 and 4. Reference numerals 271-274 indicate serpentine bands 20 wherein the description of the embodiments of FIGS. 3 and 4 may be applied to the embodiment of FIG. 6.

The component length l of a serpentine band 20 as measured in a stent lengthwise direction may increase as the distance away from the center of the side branch structure 42 along the length of the stent 10 increases, for example as described herein with respect to FIGS. 4 and 5. Reference numerals 371-374 indicate serpentine bands 20 wherein the description of the embodiments of FIGS. 3 and 4 may be applied to the embodiment of FIG. 6.

In some embodiments, the stent 10 may comprise a central serpentine band 80 that may be located midway along the length of the stent 10. A first proximal band 82 may be located proximal to the central serpentine band 80, and a first distal band 84 may be located distal to the central serpentine band 80. In some embodiments, the first distal band 84 may comprise a substantial mirror image of the first proximal band 82. In some embodiments, all of the serpentine bands 20 and connector struts 16 located distal to the first distal band 84 may comprise a substantial mirror image of the serpentine bands 20 and connector struts 16 located proximal to the first proximal band 82. The mirror image may be taken across a mirror image line 58 that passes through the center of the side branch structure 42. In some embodiments, the mirror image line 58 may bisect the component length l of the central serpentine band 80.

The inventive stents 10 may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The inventive stents may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments the at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

In some embodiments, the invention is directed to methods of making stents having the features described herein.

In some embodiments, the invention is directed to stents as described in the following numbered paragraphs.

1) A stent comprising:

a plurality of interconnected strut members defining a plurality of cells, a portion of the interconnected strut members comprising a side branch structure defining a side branch cell, the side branch cell being shaped differently than other cells of the stent;

the stent comprising a first end portion, a middle portion and a second end portion, the side branch structure located in the middle portion;

the interconnected strut members further defining a plurality of serpentine bands and a plurality of connector struts, adjacent serpentine bands connected by at least one connector strut;

a first unit area located in the middle portion, the first unit area including at least two interconnected strut members;

a second unit area located outside of the middle portion, the second unit area having a size and shape similar to the first unit area;

wherein the first unit area includes more metal than the second unit area.

2) The stent of paragraph 1, wherein a total outer surface area of the interconnected strut members in the first unit area is greater than a total outer surface area of the interconnected strut members in the second unit area.

3) The stent of paragraph 1, wherein the first unit area comprises at least one connector strut and at least a portion of two serpentine bands.

4) The stent of paragraph 1 further comprising a plurality of cells, wherein a total area of cells in the first unit area is less than a total area of cells in the second unit area.

5) The stent of paragraph 1, wherein the first end portion, the middle portion and the second end portion have equal lengths.

6) The stent of paragraph 1, wherein the first unit area includes at least one strut of the side branch structure.

7) The stent of paragraph 1, wherein the second unit area is located in the second end portion.

8) The stent of paragraph 1, further comprising a third unit area having a size and shape similar to the first unit area, wherein the first unit area includes more metal than the third unit area.

9) The stent of paragraph 8, wherein the second unit area includes more metal than the third unit area.

10) The stent of paragraph 1, further comprising a third unit area having a size and shape similar to the first unit area, wherein a total outer surface area of the interconnected strut members in the first unit area is greater than a total outer surface area of the interconnected strut members in the third unit area.

11) The stent of paragraph 10, wherein a total outer surface area of the interconnected strut members in the second unit area is greater than a total outer surface area of the interconnected strut members in the third unit area.

12) The stent of paragraph 10, wherein the second unit area is located between the first unit area and the third unit area along the length of the stent.

13) The stent of paragraph 10, wherein the second unit area and the third area are located on opposite sides of the side branch structure along the length of the stent.

14) The stent of paragraph 1, further comprising a third unit area having a size and shape similar to the first unit area, wherein a total outer surface area of the interconnected strut members in the first unit area is substantially equal to a total outer surface area of the interconnected strut members in the third unit area.

15) The stent of paragraph 14, wherein the second unit area and the third area are located on opposite sides of the side branch structure along the length of the stent.

16) The stent of paragraph 14, wherein the interconnected strut members of the third unit area comprise a substantial mirror image of the interconnected strut members of the second unit area.

17) The stent of paragraph 16, wherein the substantial mirror image is taken across a line that passes through a center of the side branch structure.

18) The stent of paragraph 1, wherein a first serpentine band located in the middle portion is connected to a second serpentine band by a first connector column comprising a plurality of connector struts; a third serpentine band located outside the middle portion is connected to a fourth serpentine band by a second connector column comprising at least one connector strut; the first connector column having more connector struts than the second connector column.

19) The stent of paragraph 18, the stent further comprising a plurality of connector columns, wherein the first connector column is located distal to the side branch structure along the length of the stent, and each connector column located distal to the first connector column includes less connector struts than the first connector column.

20) The stent of paragraph 1, wherein a first serpentine band located in the middle portion comprises a first strut having a first width; a second serpentine band located outside of the middle portion comprises a second strut having a second width, the first width being greater than the second width.

21) The stent of paragraph 1, further comprising a first serpentine band and a second serpentine band, wherein adjacent distal valleys of the first serpentine band are separated by a first distance and adjacent distal valleys of the second serpentine band are separated by a second distance that is greater than the first distance.

22) The stent of paragraph 21, wherein the distance between adjacent distal valleys of each serpentine band located distal to the second serpentine band increases with each successive serpentine band.

23) The stent of paragraph 1, further comprising a first serpentine band and a second serpentine band, wherein the first serpentine band is located in the middle portion of the stent and comprises a first component length, the second serpentine band is located distal to the first serpentine band, and the second serpentine band comprises a second component length that is greater than the first component length.

24) The stent of paragraph 23, wherein a component length of each serpentine band located distal to the second serpentine band increases with each successive serpentine band.

25) The stent of paragraph 23, the second serpentine band comprising fewer distal valleys than the first serpentine band.

26) The stent of paragraph 25, wherein the number of distal valleys of each serpentine band located distal to the second serpentine band decreases with each successive serpentine band.

In some embodiments, stents 10 may have a varying number of connector struts 16 per connector column 19, for example as shown in FIGS. 1 and 6, and as described in the following numbered paragraphs:

1) A stent comprising:

a plurality of interconnected strut members defining a plurality of cells, a portion of the interconnected strut members comprising a side branch structure defining a side branch cell, the side branch cell being shaped differently than other cells of the stent;

the stent comprising a first end portion, a middle portion and a second end portion, the side branch structure located in the middle portion;

the interconnected strut members further defining a plurality of serpentine bands and a plurality of connector struts, adjacent serpentine bands connected by a connector column comprising at least one connector strut;

wherein a first serpentine band located in the middle portion is connected to a second serpentine band by a first connector column comprising a plurality of connector struts; a third serpentine band located outside the middle portion is connected to a fourth serpentine band by a second connector column comprising at least one connector strut; the first connector column having more connector struts than the second connector column.

2) The stent of paragraph 1, further comprising a fifth serpentine band connected to the second serpentine band by a third connector column comprising at least one connector strut; the first connector column having more connector struts than the third connector column.

3) The stent of paragraph 2, the third connector column having more connector struts than the second connector column.

4) The stent of paragraph 2, wherein the fifth serpentine band is connected to the fourth serpentine band by a fourth connector column, the fourth connector column comprising at least one connector strut.

5) The stent of paragraph 4, wherein the third connector column comprises more connector struts than the fourth connector column.

6) The stent of paragraph 5, wherein the fourth connector column comprises more connector struts than the second connector column.

7) The stent of paragraph 1, further comprising a third connector column having the same number of connector struts as the second connector column, the second connector column and the third connector column located on opposite sides of the side branch structure along the length of the stent.

8) The stent of paragraph 7, further comprising a fourth connector column having the same number of connector struts as the first connector column, the first connector column and the fourth connector column located on opposite sides of the side branch structure along the length of the stent.

9) The stent of paragraph 1, wherein the first connector column includes a straight connector strut oriented in a first direction and the third connector column includes a straight connector strut oriented in a second direction that is different than the first direction.

10) The stent of paragraph 1, wherein the second serpentine band comprises alternating straight struts and bent struts.

11) The stent of paragraph 10, wherein the third serpentine band comprises the same shape as the second serpentine band.

12) The stent of paragraph 1, wherein the first connector column is located distal to the side branch structure along the length of the stent, and each connector column located distal to the first connector column includes less connector struts than the first connector column.

In some embodiments, stents 10 may have comprise serpentine bands 20 having struts 22 with varying widths, for example as shown in FIG. 2, and as described in the following numbered paragraphs:

1) A stent comprising:

a plurality of interconnected strut members defining a plurality of cells, a portion of the interconnected strut members comprising a side branch structure defining a side branch cell, the side branch cell being shaped differently than other cells of the stent;

the stent comprising a first end portion, a middle portion and a second end portion, the side branch structure located in the middle portion;

the interconnected strut members further defining a plurality of serpentine bands and a plurality of connector struts, adjacent serpentine bands connected by a connector column comprising at least one connector strut;

wherein a first serpentine band located in the middle portion comprises a first strut having a first width; a second serpentine band located outside of the middle portion comprises a second strut having a second width, the first width being greater than the second width.

2) The stent of paragraph 1, further comprising a third serpentine band comprising a strut having a width equal to the second width, the second serpentine band and the third serpentine band located on opposite sides of the side branch structure.

3) The stent of paragraph 1, further comprising a third serpentine band located between the first serpentine band and the second serpentine band, the third serpentine band comprising a third strut having a third width, the third width being less than the first width.

4) The stent of paragraph 3, wherein the third width is greater than the second width.

5) The stent of paragraph 3, further comprising a fourth serpentine band located between the first serpentine band and the third serpentine band, the fourth serpentine band comprising a fourth strut having a fourth width, the fourth width being less than the first width.

6) The stent of paragraph 5, wherein the fourth width is greater than the third width.

7) The stent of paragraph 1, wherein the first serpentine band is located distal to the side branch structure along the length of the stent, and wherein each serpentine band that is located distal to the first serpentine band comprises a strut having a width that is less than the first width.

8) The stent of paragraph 1, wherein the first serpentine band comprises alternating straight struts and bent struts.

9) The stent of paragraph 1, wherein a first connector column comprises a first connector strut that connects to the first serpentine band, the first connector strut having a first connector strut width; and wherein a second connector column comprises a second connector strut that connects to the second serpentine band, the second connector strut having a second connector strut width, the first connector strut width being greater than the second connector strut width.

10) The stent of paragraph 9, wherein the first connector strut is oriented in a first direction and the second connector strut is oriented in a second direction that is different than the first direction.

11) The stent of paragraph 9, further comprising a third connector column comprising a third connector strut that connects to the second serpentine band, the third connector strut having a third connector strut width that is less than the second connector strut width.

12) The stent of paragraph 11, wherein the third connector strut is oriented in the same direction as the first connector strut.

In some embodiments, stents 10 may include serpentine bands 20 that comprise varying wavelengths or comprise varying numbers of proximal peaks 24 and/or distal valleys 26, for example as shown in FIGS. 3, 4 and 6, and as described in the following numbered paragraphs:

1) A stent comprising:

a plurality of interconnected strut members defining a plurality of cells, a portion of the interconnected strut members comprising a side branch structure defining a side branch cell, the side branch cell being shaped differently than other cells of the stent;

the stent comprising a first end portion, a middle portion and a second end portion, the side branch structure located in the middle portion;

the interconnected strut members further defining a plurality of serpentine bands and a plurality of connector struts, each serpentine band comprising alternating proximal peaks and distal valleys, adjacent serpentine bands connected by a connector column comprising at least one connector strut;

a first serpentine band wherein adjacent distal valleys of the first serpentine band are separated by a first distance;

a second serpentine band wherein adjacent distal valleys of the second serpentine band are separated by a second distance that is greater than the first distance.

2) The stent of paragraph 1, wherein the second serpentine band is located distal to the first serpentine band along the length of the stent.

3) The stent of paragraph 2, wherein the first serpentine band is located distal to the side branch structure along the length of the stent.

4) The stent of paragraph 1, wherein the first serpentine band connects to at least one strut of the side branch structure.

5) The stent of paragraph 1, further comprising a third serpentine band wherein adjacent distal valleys of the third serpentine band are separated by a distance that is equal to the second distance.

6) The stent of paragraph 5, wherein the second serpentine band and the third serpentine band are located on different sides of the side branch structure along the length of the stent.

7) The stent of paragraph 1, further comprising a third serpentine band wherein adjacent distal valleys of the third serpentine band are separated by a third distance that is greater than the first distance.

8) The stent of paragraph 7, wherein the second distance is less than the third distance.

9) The stent of paragraph 7, wherein the third serpentine band comprises fewer distal valleys than the second serpentine band.

10) The stent of paragraph 7, further comprising a fourth serpentine band wherein adjacent distal valleys of the fourth serpentine band are separated by a fourth distance that is greater than the third distance.

11) The stent of paragraph 10, wherein the fourth serpentine band comprises fewer distal valleys than the third serpentine band.

12) The stent of paragraph 1, wherein the second serpentine band comprises alternating straight struts and bent struts.

13) The stent of paragraph 1, wherein a first connector strut connects to a proximal peak of the second serpentine band and a second connector strut connects to a distal valley of the second serpentine band, wherein the first connector strut is oriented in a first direction and the second connector strut is oriented in a second direction that is different than the first direction.

14) The stent of paragraph 1, wherein the distance between adjacent distal valleys of each serpentine band located distal to the second serpentine band is greater than the second distance.

15) The stent of paragraph 14, wherein the distance between adjacent distal valleys of each serpentine band located distal to the second serpentine band increases with each successive serpentine band.

In some embodiments, stents 10 may include serpentine bands 20 that comprise varying component lengths as measured parallel to a stent lengthwise axis 11, or comprise struts 22 of varying lengths, for example as shown in FIGS. 4, 5 and 6, and as described in the following numbered paragraphs:

1) A stent comprising:

a plurality of interconnected strut members defining a plurality of cells, a portion of the interconnected strut members comprising a side branch structure defining a side branch cell, the side branch cell being shaped differently than other cells of the stent;

the stent comprising a first end portion, a middle portion and a second end portion, the side branch structure located in the middle portion;

the interconnected strut members further defining a plurality of serpentine bands and a plurality of connector struts, adjacent serpentine bands connected by at least one connector strut;

each serpentine band comprising alternating struts and turns, each serpentine band having a component length comprising the distance along the length of the stent spanned by the serpentine band;

a first serpentine band located in the middle portion of the stent having a first component length;

a second serpentine band located distal to the first serpentine band, the second serpentine band having a second component length that is greater than the first component length.

2) The stent of paragraph 1, further comprising a third serpentine band having a third component length that is equal to the second component length.

3) The stent of paragraph 2, wherein the second serpentine band and the third serpentine band are located on different sides of the side branch structure along the length of the stent.

4) The stent of paragraph 1, further comprising a third serpentine band having a third component length that is greater than the second component length.

5) The stent of paragraph 4, wherein the second serpentine band is located between the first serpentine band and the third serpentine band along the length of the stent.

6) The stent of paragraph 4, wherein the struts of the third serpentine band are longer than the struts of the second serpentine band.

7) The stent of paragraph 6, wherein the turns of the second serpentine band and the turns of the third serpentine band comprise the same size and shape.

8) The stent of paragraph 4, further comprising a fourth serpentine band having a fourth component length that is greater than the third component length.

9) The stent of paragraph 8, wherein the fourth serpentine band is located distal to the third serpentine band along the length of the stent.

10) The stent of paragraph 1, wherein the first serpentine band connects to the side branch structure.

11) The stent of paragraph 1, wherein the second serpentine band is located distal to the side branch structure.

12) The stent of paragraph 11, wherein the component length of each serpentine band located distal to the second serpentine band increases with each successive serpentine band.

13) The stent of paragraph 1, wherein the struts of the second serpentine band comprise alternating straight struts and bent struts.

In some embodiments, stents 10 may include serpentine bands 20 that comprise varying wavelengths or comprise varying numbers of proximal peaks 24 and/or distal valleys 26, and that may comprise varying component lengths as measured parallel to a stent lengthwise axis 11, or may comprise struts 22 of varying lengths, for example as shown in FIGS. 4 and 6, and as described in the following numbered paragraphs:

1) A stent comprising:

a plurality of interconnected strut members defining a plurality of cells, a portion of the interconnected strut members comprising a side branch structure defining a side branch cell, the side branch cell being shaped differently than other cells of the stent;

the stent comprising a first end portion, a middle portion and a second end portion, the side branch structure located in the middle portion;

the interconnected strut members further defining a plurality of serpentine bands and a plurality of connector struts, adjacent serpentine bands connected by at least one connector strut;

each serpentine band comprising struts connected by alternating proximal peaks and distal valleys, each serpentine band having a component length comprising the distance along the length of the stent spanned by the serpentine band;

a first serpentine band located in the middle portion of the stent having a first component length;

a second serpentine band located distal to the first serpentine band, the second serpentine band having a second component length that is greater than the first component length, the second serpentine band comprising fewer distal valleys than the first serpentine band.

2) The stent of paragraph 1, further comprising a third serpentine band, a component length of the third serpentine band equal to the second component length.

3) The stent of paragraph 2, wherein the third serpentine band comprises the same number of distal valleys as the second serpentine band.

4) The stent of paragraph 3, wherein the second serpentine band and the third serpentine band are located on different sides of the side branch structure along the length of the stent.

5) The stent of paragraph 1, further comprising a third serpentine band having a third component length that is greater than the second component length.

6) The stent of paragraph 5, wherein the third serpentine band comprises fewer distal valleys than the second serpentine band.

7) The stent of paragraph 5, further comprising a fourth serpentine band having a fourth component length that is greater than the third component length.

8) The stent of paragraph 7, wherein the fourth serpentine band comprises fewer distal valleys than the third serpentine band.

9) The stent of paragraph 7, wherein the third serpentine band is located between the second serpentine band and the fourth serpentine band.

10) The stent of paragraph 1, wherein the struts of the second serpentine band comprise alternating straight struts and bent struts.

11) The stent of paragraph 1, wherein a component length of each serpentine band located distal to the second serpentine band increases with each successive serpentine band.

12) The stent of paragraph 1, wherein the number of distal valleys of each serpentine band located distal to the second serpentine band decreases with each successive serpentine band.

In some embodiments, the invention is directed to methods of making stents as described in the previous numbered paragraphs.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1) A stent comprising:
a plurality of interconnected strut members defining a plurality of cells, a portion of the interconnected strut members comprising a side branch structure defining a side branch cell, the side branch cell being shaped differently than other cells of the stent;
the stent comprising a first end portion, a middle portion and a second end portion, the side branch structure located in the middle portion;
the interconnected strut members further defining a plurality of serpentine bands and a plurality of connector struts, adjacent serpentine bands connected by at least one connector strut;
a first unit area located in the middle portion, the first unit area including at least two interconnected strut members;
a second unit area located outside of the middle portion, the second unit area having a size and shape similar to the first unit area;
wherein a total outer surface area of the interconnected strut members in the first unit area is greater than a total outer surface area of the interconnected strut members in the second unit area.

2) The stent of claim 1, wherein the first unit area comprises at least one connector strut and at least a portion of two serpentine bands.

3) The stent of claim 1, wherein the first end portion, the middle portion and the second end portion have equal lengths.

4) The stent of claim 1, wherein the first unit area includes at least one strut of the side branch structure.

5) The stent of claim 1, further comprising a third unit area having a size and shape similar to the first unit area, wherein a total outer surface area of the interconnected strut members in the first unit area is greater than a total outer surface area of the interconnected strut members in the third unit area.

6) The stent of claim 5, wherein a total outer surface area of the interconnected strut members in the second unit area is greater than a total outer surface area of the interconnected strut members in the third unit area.

7) The stent of claim 5, wherein the second unit area is located between the first unit area and the third unit area along the length of the stent.

8) The stent of claim 5, wherein the second unit area and the third area are located on opposite sides of the side branch structure along the length of the stent.

9) The stent of claim 1, wherein a first serpentine band located in the middle portion is connected to a second serpentine band by a first connector column comprising a plurality of connector struts; a third serpentine band located outside the middle portion is connected to a fourth serpentine band by a second connector column comprising at least one connector strut; the first connector column having more connector struts than the second connector column.

10) The stent of claim 1, wherein a first serpentine band located in the middle portion comprises a first strut having a first width; a second serpentine band located outside of the middle portion comprises a second strut having a second width, the first width being greater than the second width.

11) The stent of claim 1, further comprising a first serpentine band and a second serpentine band, wherein adjacent distal valleys of the first serpentine band are separated by a first distance and adjacent distal valleys of the second serpentine band are separated by a second distance that is greater than the first distance.

12) The stent of claim 1, further comprising a first serpentine band and a second serpentine band, wherein the first serpentine band is located in the middle portion of the stent and comprises a first component length, the second serpentine band is located distal to the first serpentine band, and the second serpentine band comprises a second component length that is greater than the first component length.

13) The stent of claim 1, wherein a first serpentine band located in the middle portion is connected to a second serpentine band by a first connector column comprising a plurality of connector struts; a third serpentine band located outside the middle portion is connected to a fourth serpentine band by a second connector column comprising at least one connector strut; the first connector column having more connector struts than the second connector column.

14) The stent of paragraph 13, further comprising a fifth serpentine band connected to the second serpentine band by a third connector column comprising at least one connector strut; the first connector column having more connector struts than the third connector column.

15) The stent of paragraph 13, further comprising a third connector column having the same number of connector struts as the second connector column, the second connector column and the third connector column located on opposite sides of the side branch structure along the length of the stent.

16) The stent of paragraph 15, further comprising a fourth connector column having the same number of connector struts as the first connector column, the first connector column and the fourth connector column located on opposite sides of the side branch structure along the length of the stent.

17) The stent of paragraph 13, wherein the first connector column includes a straight connector strut oriented in a first direction and the third connector column includes a straight connector strut oriented in a second direction that is different than the first direction.

18) The stent of paragraph 13, wherein the second serpentine band comprises alternating straight struts and bent struts.

19) The stent of paragraph 13, wherein the first connector column is located distal to the side branch structure along the length of the stent, and each connector column located distal to the first connector column includes less connector struts than the first connector column.

20) A stent comprising:
a plurality of interconnected strut members defining a plurality of cells, a portion of the interconnected strut members comprising a side branch structure defining a side branch cell, the side branch cell being shaped differently than other cells of the stent;
the stent comprising a first end portion, a middle portion and a second end portion, the side branch structure located in the middle portion;
the interconnected strut members further defining a plurality of serpentine bands and a plurality of connector struts, adjacent serpentine bands connected by at least one connector strut;
a first unit area located in the middle portion, the first unit area including at least two interconnected strut members;
a second unit area located outside of the middle portion, the second unit area having a size and shape similar to the first unit area;
wherein the first unit area includes more metal than the second unit area.
